(19) United States
(12) Patent Application Publication
(10) Pub. No.: US 2002/0055117 A1
(43) Pub. Date: May 9, 2002

(54) Methods for detecting prostate cancer

(76) Inventors: James Fett, Waltham, MA (US); Karen A. Olson, Brookline, MA (US)

Correspondence Address: John P. Iwanicki, BANNER & WITCOFF, LTD., 28th Floor, 28 State Street, Boston, MA, 02109, US

(21) Appl. No.: 09/957,376

(22) Filed: Sep. 20, 2001

Related U.S. Application Data
(60) Provisional application No. 60/234,386, filed on Sep. 20, 2000.

(51) Int. Cl. C12Q 1/68; G01N 33/574

(52) U.S. Cl. 435/6; 435/7.23

(57) ABSTRACT

Methods for detecting, diagnosing, and monitoring cancerous conditions and/or metastatic diseases such as prostate cancer are disclosed. Angiogenin expression is used to identify cancerous conditions and/or metastatic diseases such as prostate cancer.

### RELATED APPLICATIONS

This application claims priority to U.S. Provisional Patent Application No. 60/234,386 filed on Sep. 20, 2000, hereby incorporated by reference in its entirety for all purposes.

This application was funded in part by the National Institutes of Health Grant No. R01 CA60046.

### BACKGROUND OF THE INVENTION

1. Field of the Invention

Embodiments of the present invention relate in general to the detection, diagnosis, and monitoring of metastatic diseases. More preferably, embodiments of the present invention relate to the detection, diagnosis, and monitoring of prostate cancer.

2. Description of Related Art

Angiogenesis, the development of new capillaries from preexisting blood vessels in normal and in malignant tissues, occurs in a variety of normal and pathological conditions including embryonic development, wound healing, rheumatoid diseases, diabetic retinopathies, and tumor growth (Folkman and Cotran, Int. Rev. Exp. Path., 16:207-248 (1976); and Folkman and Klagsburn, Science, 235:442-447 (1987), hereby incorporated by reference in their entirety for all purposes). Under normal physiological conditions, neovascularization is highly regulated by numerous stimulatory and inhibitory factors.

When regulation of angiogenesis fails, such as in certain diseases, persistent and unregulated formation of pathological capillaries results. Angiogenesis is indispensable for 30 tumor growth and metastasis (Montesano et al., Proc. Natl. Acad. Sci. USA, 83:7297-7301 (1986), hereby incorporated by reference in its entirety for all purposes). Although small tumors (1-2 mm in diameter) can receive nutrients and remove the waste products by diffusion (Thomas et al., Proc. Natl. Acad. Sci. USA, 82:6409-6413 (1985), hereby incorporated by reference in its entirety for all purposes), further growth depends on persistent angiogenesis induced by several angiogenic molecules that are released by tumor cells and normal host cells. Due to numerous reasons, such as leakage of newly formed blood vessels in tumors, malignant cells can be discharged into the circulation resulting in metastasis of the malignant cells. These discharged cells can colonize at a distant site and induce further angiogenesis for growth.

Angiogenin (ANG) protein is a 14.1 kDa cationic, single chain, secreted protein that induces angiogenesis in classical assays. Specifically it has been found to induce angiogenesis in chicken chorioallantoic membrane and rabbit cornea and meniscus. ANG protein was first isolated in the 1980s in the course of a search for tumor angiogenesis agents (Fett et al., Biochemistry, 24:5480-5486 (1985), hereby incorporated by reference in its entirety for all purposes). It is a member of the RNase family of proteins and is the only ribonuclease that elicits angiogenic activity. ANG protein is secreted by a wide range of human tumor cell types in both culture and in vivo, including those of prostate origin. ANG protein supports both endothelial and tumor cell adhesion and also stimulates endothelial cell activities appropriate for angiogenesis, such as proteolytic activity, invasiveness, and proliferation, for example. Binding of ANG protein to endothelial cells also activates the enzyme phospholipase C (PLC—an enzyme that causes the degradation of phospholipids). A striking feature of ANG protein is that it normally circulates in human plasma at a concentration of 250 to 360 ng/mL. Plasma ANG protein may act to promote wound healing when it leaves blood vessels as a result of tissue trauma, for example.

Several strategies have been investigated to block angiogenesis. One strategy includes blocking of the uptake of the angiogenic factors by the cells. In this method, one or more compounds, such as peptide antagonists, that bind to the receptors of the cells are administered. Any angiogenic factors are prevented from binding to the cell. Other methods that have targeted the action of ANG include administration of PLC antagonists such as neomycin (Hu, Proc. Natl. Acad. Sci. USA, 95:9791-9795 (1998), hereby incorporated by reference in its entirety for all purposes). Preclinical mouse studies have indicated that inhibitors of ANG function can dramatically interfere with the establishment and metastatic spread of several human tumor types including those of prostate origin (Olson et al., Cancer Res., 54:4576-4579 (1994); Olson et al., Proc. Natl. Acad. Sci. USA, 92:442-446 (1995); Olson and Fett, Proc. Am. Assoc. Cancer Res., 39:665A (1998); and Olson et al., Clin. Cancer Res., In press (2001), hereby incorporated by reference in their entirety for all purposes). It has been shown that injection of anti-ANG agents, such as an antisense drug, can reduce the metastasis of prostate cancer (see FIG. 1a). Similar results are observed when a monoclonal antibody is used in place of the antisense antibody (see FIG. 1b).

However, in addition to methods for treating metastatic diseases, a need exists to develop methods for the detection and/or diagnosis of metastatic diseases, such as prostate cancer.

### BRIEF SUMMARY OF THE INVENTION

Embodiments of the present invention are based in part on the detection of ANG DNA, ANG mRNA, or ANG protein in cancerous conditions and/or metastatic diseases such as prostate cancer. Embodiments of the present invention also include methods for screening for cancerous conditions and/or metastatic diseases, such as prostate cancer. The detection of levels of ANG protein, ANG mRNA, or ANG DNA in fluid and tissue samples of men is useful as a diagnostic/prognostic marker for prostate cancer. Detection is useful for staging of prostate cancer and as an indicator of the presence of minimal residual disease or tumor relapse. ANG levels may also be used to monitor efficacy of treatment. Embodiments of the present invention also include methods for screening test compounds that are useful for inhibiting cancerous conditions and/or metastatic diseases. Methods of the present invention may be advantageously applied and adapted for diagnostic use (e.g., in kits), such as an immunoassay, ELISA, or the like, to detect ANG protein levels in the urine and serum of men with various degrees of prostate cancer. Adaptation of this method to a diagnostic assay, provides another test for screening men for prostate cancer, in addition to the current screening methods that quantitate PSA levels.

In accordance with preferred embodiments, a method is provided to detect the presence of ANG DNA in biological samples. Preferably, the method detects the presence of genes that may express ANG after alteration of the cellular phenotype, such as in prostate cancer. The method may be used in combination with well-known molecular biology techniques, such as PCR (polymerase chain reaction) and microarray technology, to detect ANG and ANG-like genes that are present in a biological sample, such as normal and cancerous prostate tissue.

In accordance with other preferred embodiments, a method is provided to detect the presence of ANG RNA in biological samples. Preferably, the method detects the presence of ANG RNA within cells and within urine, serum, or tissue samples. The method may be used in combination with well-known molecular biology techniques, such as RT-PCR and microarray technology, to detect ANG RNA that is present in a biological sample, such as normal and cancerous prostate tissue.

In accordance with other preferred embodiments, a method is provided to detect the presence of ANG protein in biological samples. Preferably, the method detects the presence of ANG protein within cells and within urine, serum, or tissue samples. The method may be used to measure the amount of ANG protein that is present in various metastatic diseases such as prostate cancer.

In accordance with other preferred embodiments, a method is provided for detecting ANG protein in immunohistochemical analyses. Immunohistochemical studies may provide for the cellular localization of ANG protein. Additionally, the amount of ANG protein present in a cell and the types of cells where ANG protein is found may be determined.

In accordance with other preferred embodiments, a method is provided for screening urine samples for ANG protein. Numerous strategies known to those skilled in the art may be used for identifying the ANG protein including but not limited to immunoassay, gel electrophoresis, ELISA, or the like. Preferably, ELISA is used to identify the amount of ANG protein present in urine samples. Therefore, this method can be used as a rapid method for screening patients for prostate cancer.

In accordance with certain embodiments of the present invention, amounts or levels of ANG DNA, ANG mRNA or ANG protein detected according to the methods described herein can then be compared with a database of amounts or levels of ANG DNA, ANG mRNA or ANG protein associated with known metastatic conditions as a method of diagnosing certain metastatic conditions or the staging of certain metastatic conditions. Such a database can be developed according to the methods of the present invention for various metastatic conditions.

In accordance with certain embodiments of the present invention, a method is provided to monitor a cancerous condition or a metastatic disease in a subject. In one embodiment, an amount of angiogenin protein, angiogenin RNA, or angiogenin DNA present is detected in a first biological sample obtained from the subject and compared with the amount of angiogenin protein, angiogenin RNA, or angiogenin DNA present in a second biological sample obtained from the subject. The amount of angiogenin protein, angiogenin RNA, or angiogenin DNA present in the first biological sample may then be compared with the amount of angiogenin protein, angiogenin RNA, or angiogenin DNA present second sample in order to monitor a cancerous condition or metastatic disease.

In accordance with certain embodiments of the present invention, a method is provided to diagnose a cancerous condition or a metastatic disease in a subject. In one embodiment, an amount of angiogenin protein, angiogenin RNA, or angiogenin DNA present is identified in a first biological sample obtained from the subject and compared with the amount of angiogenin protein, angiogenin RNA, or angiogenin DNA present in a second biological sample obtained from the subject. The amount of angiogenin protein, angiogenin RNA, or angiogenin DNA present in the first biological sample may then be compared with the amount of angiogenin protein, angiogenin RNA, or angiogenin DNA present second sample in order to diagnose a cancerous condition or metastatic disease.

In accordance with certain embodiments of the present invention, a method is provided to prognose a cancerous condition or a metastatic disease in a subject. In one embodiment, an amount of angiogenin protein, angiogenin RNA, or angiogenin DNA present is identified in a first biological sample obtained from the subject and compared with the amount of angiogenin protein, angiogenin RNA, or angiogenin DNA present in a second biological sample obtained from the subject. The amount of angiogenin protein, angiogenin RNA, or angiogenin DNA present in the first biological sample may then be compared with the amount of angiogenin protein, angiogenin RNA, or angiogenin DNA present second sample in order to prognose a cancerous condition or metastatic disease.

In accordance with certain embodiments of the present invention, methods are provided to assess the efficacy of a test compound or a therapy for inhibiting a cancerous condition or a metastatic disease in a subject. In one embodiment, an amount of angiogenin protein, angiogenin RNA, or angiogenin DNA present in a first biological sample is obtained from a subject exposed to or maintained in a test compound and compared with the amount of angiogenin protein, angiogenin RNA, or angiogenin DNA present in a second biological sample from the subject. A reduced amount of angiogenin protein, angiogenin RNA, or angiogenin DNA present in the first biological sample relative to the second biological sample, indicates that the test compound or therapy is efficacious for inhibiting a cancerous condition in the subject.

In accordance with certain embodiments of the present invention, a method is provided to select a composition for inhibiting a cancerous condition or a metastatic disease in a subject. In one embodiment, a sample of cells is obtained from the subject, aliquots of the sample are maintained separately in the presence of a plurality of test compositions, and the expression of angiogenin protein, angiogenin RNA, or angiogenin DNA in each of the aliquots is compared. A test composition which induces a reduced amount of angiogenin protein, angiogenin RNA, or angiogenin DNA in the aliquot containing that test composition, relative to other test compositions, is selected as a composition useful for inhibiting a cancerous condition or a metastatic disease in a subject.

In accordance with certain embodiments of the present invention, a method is provided for treating a cancerous condition or a metastatic disease in a subject. In one embodiment, a compound is administered to a subject such that the amount of angiogenin protein, angiogenin RNA, or angiogenin DNA in the patient is reduced.

In accordance with certain embodiments of the present invention, a kit is provided for assessing whether a subject is afflicted with a cancerous condition or a metastatic disease in a subject. In one embodiment, the kit comprises reagents for assessing the amount of angiogenin protein, angiogenin RNA, or angiogenin DNA present in a sample.

These and other objects and aspects of the technology disclosed here, and of preferred embodiments of such technology, will be understood from the following disclosure and detailed description.

### BRIEF DESCRIPTION OF DRAWINGS

The foregoing and other features and advantages of the present invention will be more fully understood from the following detailed description of illustrative embodiments taken in conjunction with the accompanying drawings in which:

FIG. 1a shows the results of treatment (in mice) with an antisense drug. FIG. 1a shows a PBS control, concurrent administration of the antisense drug with human prostate tumor cell injection (Antisense, Day 0), administration of the antisense drug 14 days after injection of human prostate tumor cells (Antisense, day 14), and administration of the antisense drug 28 days after human tumor cell injection (Antisense, day 28).

FIG. 1b shows the results of treatment with a monoclonal antibody. FIG. 1b shows a PBS control, concurrent administration of a monoclonal antibody with human prostate tumor cell injection (mAb 26-2F, day 0), administration of a monoclonal antibody 14 days after human tumor cell injection (mAb 26-2F, day 14), and administration of a monoclonal antibody 28 days after human tumor cell injection (mAb 26-2F, day 28).

FIG. 2 shows a Northern blot analysis of normal liver tissue which verifies the presence of ANG RNA in normal liver tissue.

FIG. 3 shows a Northern blot analysis of normal prostate tissue showing that ANG RNA is present in normal prostate tissue.

FIG. 4 shows a dot blot for a matched tumor/normal expression array. The cDNAs were hybridized with a labeled probe for ANG to measure the relative message abundance present in normal tissue and tumor tissue.

FIG. 5 shows the results of RT-PCR on RNA from an LNCaP cell line using primers designed for ANG. The experiment was performed to verify that a specific primer pair was operative.

FIG. 6 shows the results of PCR on prostate matched pairs. cDNAs (those in FIG. 4) for prostate matched pairs were used to quantitate the ANG message present in two patients. T represents prostate tumor samples and N represents normal prostate samples.

FIG. 7 shows the results of a microarray comparing PC-3M to PC-3 cell gene expression. The arrow in the lower gel points to ANG RNA indicating that the ANG gene is upregulated in metastatic PC-3M cells when compared with PC-3 cells.

FIG. 8 shows the results of an array for PC-3 cell gene expression. The figure shows the total RNA isolated from PC-3 cells that has interacted with two custom arrays comprising thirty-seven different angiogenesis-related genes spotted in duplicate. The ANG gene (denoted by arrow) is present in high abundance.

FIG. 9 shows the results of immunohistochemical staining for the ANG protein of clinical samples of prostate intraepithelial neoplasia (FIG. 9a) and full-blown prostate adenocarcinoma (FIG. 9b).

FIG. 10 shows ANG mRNA and protein levels in extracts from PC-3M tumors in the prostates of control- and ANG antisense-treated athymic mice as well as serum ANG protein levels in mice in this same experiment.

FIG. 11 shows the urine ANG protein levels from normal men and men having prostate cancer.

FIG. 12 shows the urine ANG protein and serum PSA levels from men with previously diagnosed (>1 year) prostate cancer.

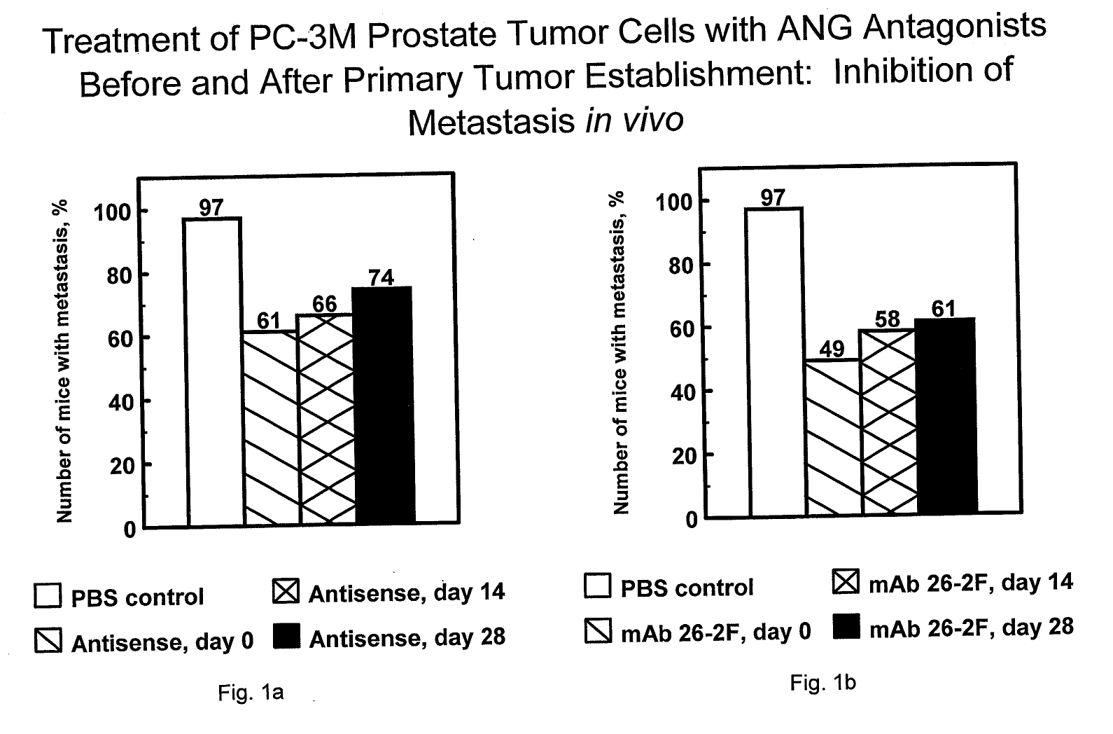

### DETAILED DESCRIPTION OF CERTAIN PREFERRED EMBODIMENTS

Numerous aspects in accordance with preferred embodiments are possible and will be apparent to those skilled in the art given the benefit of this disclosure. The detailed description herein, for convenience, will focus on certain illustrative and exemplary embodiments.

In accordance with preferred embodiments, a method for screening and diagnosing metastatic diseases is disclosed. This method may be used and adapted to numerous embodiments relating to the detection, diagnosis, and monitoring of metastatic diseases involving ANG DNA, ANG RNA, or ANG protein. Preferably, the method is used for detecting, diagnosing, and monitoring prostate cancer. The method comprises the detection, identification and quantification of ANG DNA, ANG RNA, or ANG protein within biological samples including urine samples, tissue samples, or the like.

The practice of the present invention may employ conventional techniques of molecular biology, cell biology, biochemistry, and immunology, which are within the skill of the art. Such conventional techniques include, for example, polymerase chain reaction (PCR), reverse transcription polymerse chain reaction (RT-PCR), hybridization of nucleic acid probes, detection of probe hybridization, and RNA and DNA isolation techniques. Specific illustrations of suitable techniques are presented in the examples below. However, other equivalent conventional procedures can, of course, also be used. Such conventional techniques can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), all of which are herein incorporated in their entirety by reference for all purposes.

In accordance with preferred embodiments, the level of expression of angiogenin is assessed by detecting the presence of an angiogenin nucleic acid (e.g., DNA and/or RNA) or an angiogenin protein in the sample. Detection involves contacting a sample with a compound or an agent capable of detecting an angiogenin DNA, an angiogenin RNA, or an angiogenin protein such that the presence of an angiogenin DNA, an angiogenin RNA, or an angiogenin protein is detected in the biological sample. A preferred agent for detecting angiogenin RNA or angiogenin DNA is a labeled or labelable nucleic acid probe capable of hybridizing to angiogenin RNA or angiogenin DNA. The nucleic acid probe can be, for example, complementary to angiogenin RNA or angiogenin DNA, or a portion thereof, such as an oligonucleotide which specifically hybridizes angiogenin RNA or angiogenin DNA.

A preferred agent for detecting an angiogenin protein is a labeled or labelable antibody capable of binding to the angiogenin protein. Antibodies can be polyclonal, or more preferably, monoclonal. An intact antibody, antibody derivative, or a fragment thereof (e.g., Fab or F(ab′)₂) can be used. The term “labeled or labelable,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The detection methods described herein can be used to detect angiogenin DNA, angiogenin RNA, or angiogenin protein in a biological sample in vitro as well as in vivo. In vitro techniques for detection of angiogenin RNA include, but are not limited to, Northern hybridizations and in situ hybridizations. In vitro techniques for detection of angiogenin DNA include, but are not limited to, Southern hybridizations and PCR. In vitro techniques for detection of angiogenin protein include, but are not limited to, enzyme linked immunosorbent assays (ELISAs), Western blots, immunoprecipitations, and immunofluorescence assays. Alternatively, angiogenin protein can be detected in vivo in a subject by introducing into the subject a labeled antibody against the marker protein. For example, the antibody can be labeled with a radioactive marker whose presence and location in a subject can be detected by standard imaging techniques.

In a preferred embodiment, the invention provides methods of assessing the efficacy of test compounds and compositions for treating cancerous conditions and/or metastatic diseases. The methods entail identifying candidate or test compounds or agents (e.g., peptides, peptidomimetics, small molecules or other drugs) which have an inhibitory effect on cancerous conditions and/or metastatic diseases. Candidate or test compounds or agents which have an inhibitory effect on cancerous conditions and/or metastatic diseases are identified in assays that employ cancer and/or metastatic cells, such as an expression assay entailing direct or indirect measurement of the expression of angiogenin DNA, angiogenin RNA, or angiogenin protein. For example, modulators of angiogenin DNA, angiogenin RNA, or angiogenin protein expression can be identified in a method in which a cell is contacted with a candidate compound and the expression of angiogenin DNA, angiogenin RNA, or angiogenin protein in the cell is determined. The level of expression of angiogenin DNA, angiogenin RNA, or angiogenin protein in the presence of the candidate compound is compared to the level of expression of angiogenin DNA, angiogenin RNA, or angiogenin protein in the absence of the candidate compound. The candidate compound can then be identified as a modulator of expression of cancerous conditions and/or metastatic diseases based on this comparison.

The invention also encompasses kits for assessing whether a subject is afflicted with a cancerous condition and/or a metastatic disease. The kit may comprise a labeled compound or agent capable of detecting angiogenin DNA, angiogenin RNA, or angiogenin protein in a biological sample, a means for determining the amount of angiogenin DNA, angiogenin RNA, or angiogenin protein in the sample, and a means for comparing the amount of angiogenin DNA, angiogenin RNA, or angiogenin protein in the sample with a standard. The compound or agent can be packaged in a suitable container. The kit can further comprise instructions for using the kit to detect a cancerous condition and/or a metastatic disease.

### EXAMPLE I

### Method for Identifying the ANG Message

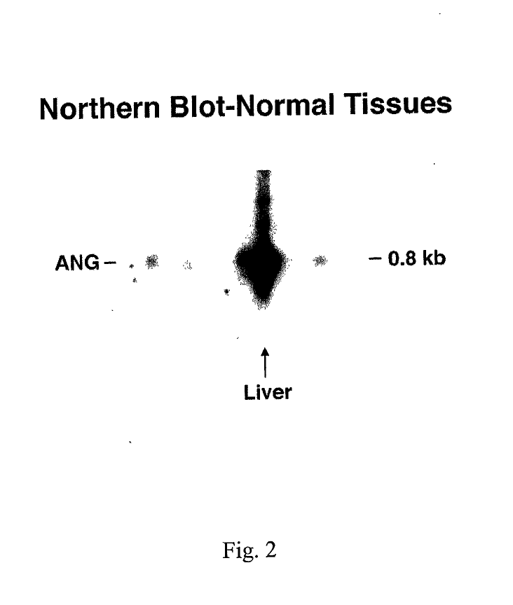

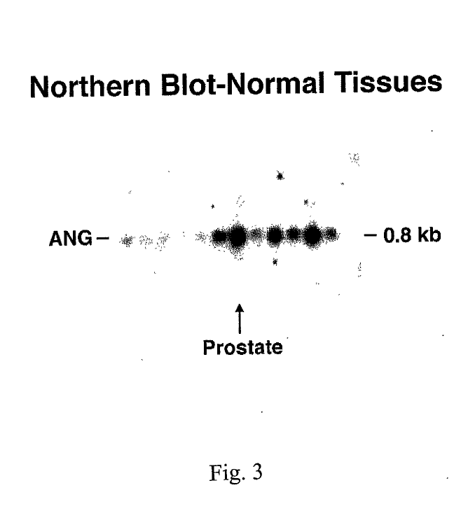

In accordance with preferred embodiments, a method for identifying the presence of the ANG message and amount of ANG message that is present in a biological sample is provided. Referring to FIG. 2, as a positive control a Northern-blot analysis was performed to identify the normal tissue profile for ANG RNA in the liver (Sambrook et al., Molecular Cloning: A Laboratory Manual (2nd ed.), Vols. 1-3, Cold Spring Harbor Laboratory, (1989), hereby incorporated by reference in its entirety for all purposes). Pre-made normal tissue Northern blots (Clonetech, Cat#7780-1) were hybridized to an ANG probe. Protocols for performing the Northern blot were followed as directed in “Multiple Tissue Northern Blot User Manual (PT1200-1)” which is available from Clonetech Laboratories, Inc., Palo Alto, Calif. The ANG cDNA probe was constructed using the methods of Weremowicz et al., Am. J. Hum. Genet., 47:973-981 (1990) (hereby incorporated by reference in its entirety for all purposes) and was labeled with [α-P32] dCTP using a “DECAprime DNA Labeling Kit” and instructions from Ambion, Inc., Austin, Tex. A very strong band was apparent at 0.8 kb that corresponds to the mRNA for ANG in the liver. This result is consistent with known information about ANG abundance in the body. ANG is a plasma protein that normally circulates throughout the body, but the liver is a primary source for synthesis of ANG. Expression of the ANG message was also detected in the normal prostate gland tissue, as visualized using a Northern-blot analysis (Clonetech, Cat# 7784-1) (see FIG. 3).

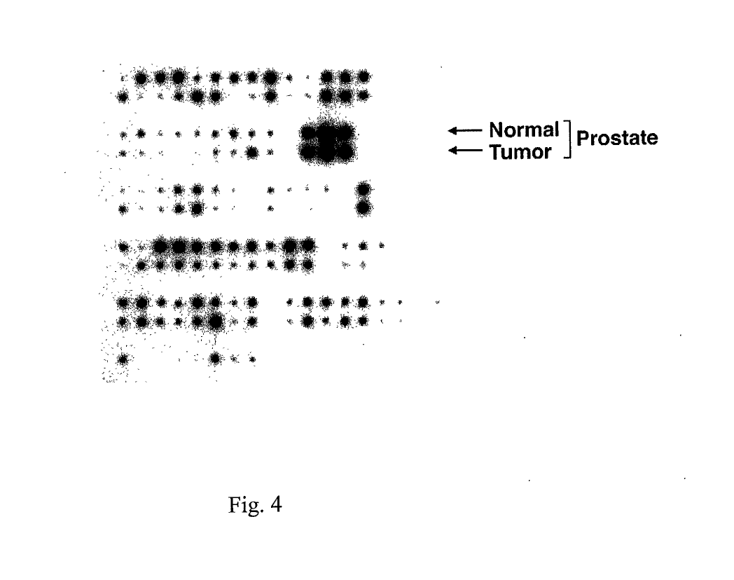

Investigation of cell types that contain the ANG message is necessary because ANG may be expressed in certain diseases characterized by alteration of a cell's phenotype (i.e., cancer), even though many cell types may not express ANG when in their normal state. To investigate the relative message abundance for a specific gene of interest, a tumor/normal cell expression array (Clonetech Cat# 7840-1) was used in which cDNAs synthesized from 68 human tumors and corresponding normal tissue from the same individual were immobilized as separate dots. These samples represent pairs of samples for several tissue types. The protocols for performing this analysis were followed as directed in “Matched Tumor/Normal Expression Array/User Manual (PT3424-1)” available from Clonetech. The cDNA probe was created as directed by Weremowicz et al., 1990. Both normal and tumor cDNAs from prostate patients were hybridized with a labeled probe for ANG. Strong signals were observed in the case of both normal and tumor samples from these patients (see. FIG. 4). The ANG message was present in both normal and cancerous prostate tissue.

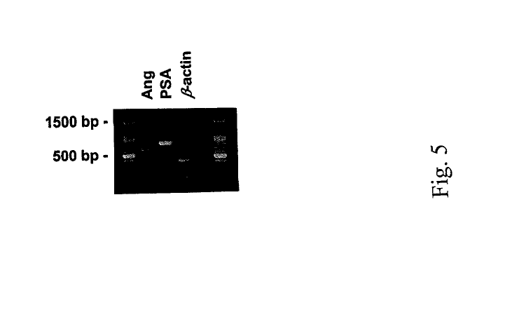

To determine whether the target ANG RNA represents a single transcript size or polymorphic mRNA, PCR (Sambrook et al., Molecular Cloning: A Laboratory Manual (2nd ed.), Vols. 1-3, Cold Spring Harbor Laboratory, (1989), hereby incorporated by reference in its entirety for all purposes). was performed on the samples shown in FIG. 4 (see arrow in FIG. 4). To achieve this goal, specific primers for ANG were designed and tested. The primers were designed using the computer program OLIGO and as described by Rychlik et al., Nucleic Acids Res., 18:6409-6412 (1990), hereby incorporated by reference in its entirety for all purposes. The primers were based on the ANG gene sequence published by Kurachi et al., Biochemistry, 33:5421-5427 (1985), hereby incorporated by reference in its entirety for all purposes. To test the primer, total RNA from the androgen-sensitive LNCaP cell line was isolated using the “Ultraspec™ RNA Isolation System” and instructions from Bioteex Laboratories, Inc. Houston, Tex. PCR was performed for ANG RNA along with reactions for the positive controls using prostate-specific antigen (PSA) and the housekeeping gene actin. RT-PCR was performed using the “GeneAmp RNA PCR Kit (Cat #N808-0017) and instructions from Perkin Elmer, Foster City, Calif. Referring to FIG. 5, the expected products were observed in each of the three lanes. There is a noticeable band at 592 bp that is characteristic of the ANG message.

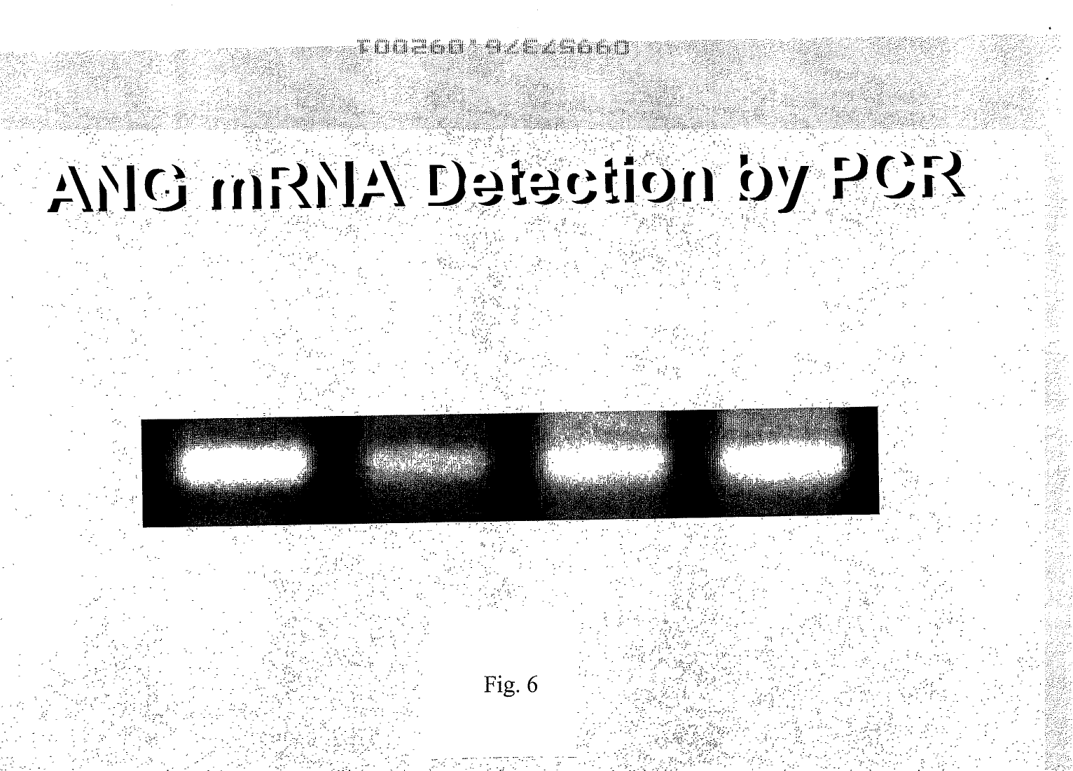

cDNAs were purchased (Clonetech Cat #HP101P and Cat#HP102P) for 2 of the 3 prostate matched pairs shown in FIG. 4. The protocol was followed in the Clonetech “Matched cDNA Pairs User Manual (PT3391-1).” PCR was performed on ANG using the protocols and primers listed above (Rychlik et al., Nucleic Acids Res., 18:6409-6412 (1990); and Kurachi et al., Biochemistry, 24:5494-5495 (1985), hereby incorporated by reference in their entirety for all purposes). The cDNAs were used to observe a PCR product with the correct molecular weight in both tumor (T) and normal (N) samples (see FIG. 6). In patient #1, the ANG message appears to be upregulated in the tumor tissue when compared with the ANG message in normal tissue. Without wishing to be bound to any scientific theory, the results appear to indicate that ANG may be upregulated in prostate cancer.

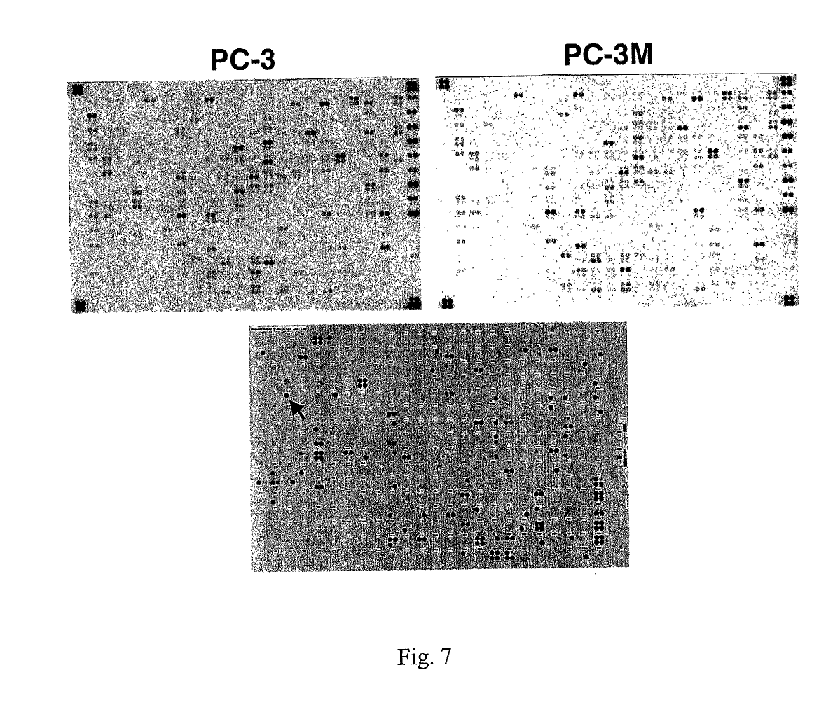

To further investigate the possible upregulation of ANG in prostate cancer, a metastatic PC-3M cell line was used to compare the amount of the ANG message present in PC-3M cells with the amount of the ANG message present in the less metastatic PC-3 parent. A nylon membrane (Sigma-Genosys) that contained a probe for ANG was used to aid in the measurement of the amount of ANG RNA that was present. The total RNA was isolated using the “UltraspecTm RNA Isolation System” and instructions from Biotecx Laboratories, Inc. Houston, Tex. The isolated RNA was treated with DNase I using Clonetech “Atlas Pure Total RNA Labeling System (Cat. #K103801).” The microarrays that were used were the “Panorama™ Human Cytokine Gene Arrays (Cat.# PRCK0002)” and “Panorama™ Human Cytokine Gene Arrays cDNA Labeling and Hybridization Kit (Cat.# PRLC0001) and the protocols were followed as directed in the manual from Sigma-Genosys, The Woodlands, TX. Recent reviews of microarray technology can be found in Planet et al., Genome Res., 11:1149-1155 (2001) and Schulze and Downward, Nat. Cell Biol., 3:E190-195 (2001), hereby incorporated by reference in their entirety for all purposes. Referring to FIG. 7, the top two patterns are the raw phosphorimager data that were converted into a color-coded composite (bottom gel in FIG. 7), which show genes that are upregulated in PC-3M cells. The arrow (see bottom gel of FIG. 7) points to the dot showing that ANG is upregulated in PC-3M cells. Without wishing to be bound by any scientific theory, it is believed that the increased amounts of the ANG message may play a role in the metastatic phenotype of prostate cancer.

### EXAMPLE II

### Angiogenesis Profiles

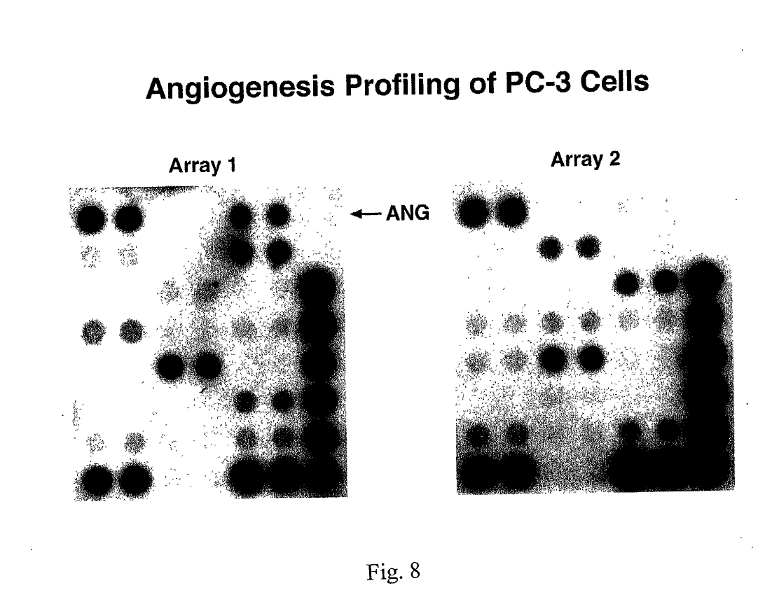

In accordance with preferred embodiments, the “angiogenesis profiles” of human prostate tumors were determined to identify the genes of this class of molecules that are differentially expressed in tumors. For example, it is possible that different ANG or ANG-like genes exist in different types of tumors and in different people. Identification of these genes may lead to the identification of numerous therapeutic targets. Since individuals may have different ANG or ANG-like genes, the therapeutics may be tailored to target the specific gene or gene product of the individual. The total RNA isolated from PC-3 cells was used to identify angiogenesis related genes. The total RNA was isolated using the “Ultraspec™ RNA Isolation System” and instructions from Biotecx Laboratories, Inc. Houston, Tex. The microarrays “Human Cancer/Angiogenesis-1 (catalog number hGEA9908030)” and “Human Cancer/Angiogenesis-2 (catalog number hGEA9911020)” and the protocols obtained from SuperArray Inc, Bethesda, Md. were used. The results of the total RNA interacting with two custom arrays carrying a total of 37 different angiogenesis-related genes spotted in duplicate are shown in FIG. 8. The strong spots at the right and bottom edges are positive controls for housekeeping genes. Referring to Array 1 of FIG. 8, the ANG message is strongly present in PC-3 cells (see arrow in Array 1 of FIG. 8). Other positive regulators of angiogenesis, such as angiopoietin-1 and VEGF-D, are also known to be expressed in this cell type. Additionally, the expression of basic fibroblast growth factor (bFGF) and the main form of vascular endothelial growth factor (VEGF) were found to be low.

The arrays shown in FIG. 8 were used to identify upregulated genes in PC-3M cells. Numerous genes encoding for angiogenesis activators may be upregulated in metastatic diseases. It is also possible that all genes, including those encoding for negative regulators of angiogenesis, are upregulated in metastatic diseases. Table 1 below shows the results of the studies performed (shown in FIG. 8) to measure the ANG RNA levels in PC-3M and PC-3 cells.

TABLE I
Upregulated Genes

| | |
|---|---|
| Gene | Expression in PC-3M compared to PC3 |

| | | |
|---|---|---|
| ANG | 2X | increase |
| Angiopoietin-1 | 2X | increase |
| Endothelin-R | 2X | increase |
| FGFR-3 | 2X | increase |
| IFN-2 alpha | 2.5X | increase |
| IL-8 | 9X | increase |
| PAI-1 | 2X | increase |
| TGF alpha | 2X | increase |
| TGF beta 2 | 3X | increase |
| TIE-2 | 2X | increase |
| uPA | 6X | increase |
| uPA-R | 3X | increase |
| VEGF | 2.5X | increase |
| VEGF-C | 3X | increase |

ANG was found to be upregulated in the metastatic cell line, as were a large number of other genes with IL-8 being the most highly upregulated gene. No genes were found to be down-regulated. Several negative regulators of angiogenesis, such as angiopoietin-2, endostatin and thrombospondins are very weakly expressed in either cell line (see Table 2).

TABLE 2
Weak or Unchanged Genes

| | |
|---|---|
| Angiopoietin-2 | PEDE |
| Endostatin | PF-4 |
| Endothelin- 1 | Prolactin |
| FGFs-2, 4 | Pleiotrophin |
| FGFR-1 | TSPs-1, 2, 3, 4 |
| FLK-1 | TIE-1 |
| FLT-1 | VEGF-B |
| HGF | VEGF-D |
| INF-beta 1 | VEGI |
| PAI-2 | |

These results indicate that angiogenesis activators are upregulated in metastatic cells while angiogenesis inhibitors remained expressed in normal amounts. Therefore, increased amounts of ANG may contribute to the prostate cancer phenotype and even higher amounts of ANG may contribute to the metastatic prostate cancer phenotype.

### EXAMPLE III

### Method for Detecting ANG Protein in Cells and Biological Samples

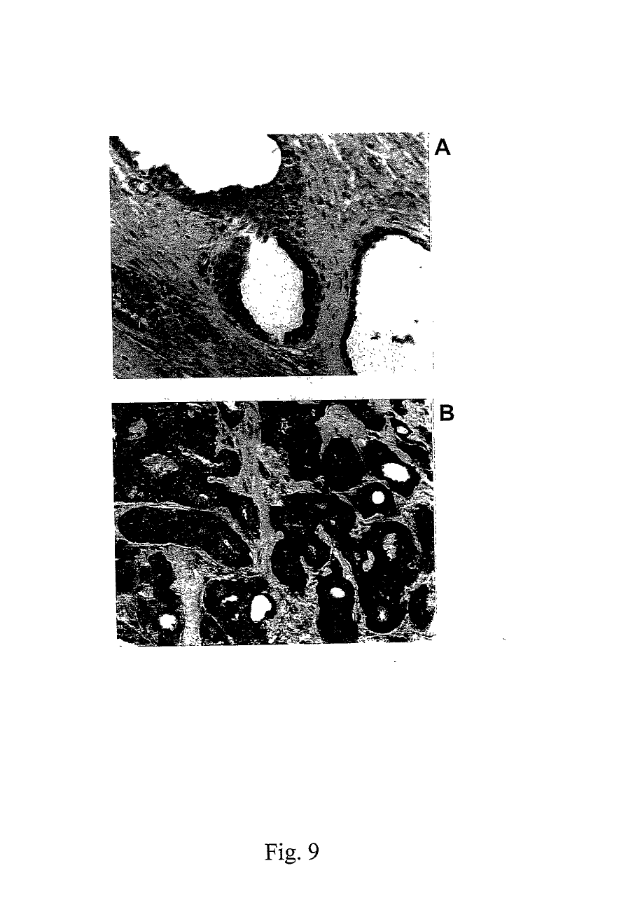

In accordance with preferred embodiments, a method for immunohistochemical studies is disclosed. The method allows for visualization and localization of ANG protein with a cell or tissue sample. Referring to FIG. 9a and FIG. 9b, prostate cells were stained with a monoclonal antibody to ANG. The monoclonal antibody 26-2F was constructed and used as described by Fett et al., Biochemistry, 33:5421-5427 (1994) and Sanno et al., Am. J. Clin. Pathol., 106:16-21 (1996), hereby incorporated by reference in their entirety for all purposes. Antigen retrieval was performed as described by Shi et al., J. Histochem. Cytochem., 39:741-748 (1991) (hereby incorporated by reference in its entirety for all purposes), and the tumor tissue was fixed as described by Olson et al., Proc. Natl. Acad. Sci. USA, 92:442-446 (1995), hereby incorporated by reference in its entirety for all purposes. The protocol for staining was followed as directed in “Dako Catalyzed Signal Amplification (CSA) System Kit (Cat. #K1500)” and the accompanying user manual. The ANG protein can be detected (darker brown regions) in cancerous secretory cells lining the ducts of the prostate, such as in prostate intraepithelial neoplasia (PIN) (see FIG. 9a), and in virtually all tumor cells in full-blown adenocarcinoma of the prostate (see FIG. 9b). ANG can also be detected in normal prostate tissue (results not shown), but as shown above in FIG. 6, the amount of the ANG message in normal prostate tissue is reduced when compared to the amount of the ANG message in cancerous prostate tissue. Therefore, since the ANG message appears to be upregulated in prostate cancer, especially in metastatic prostate cancer, ANG protein may be used as an effective marker for identification of prostate cancer.

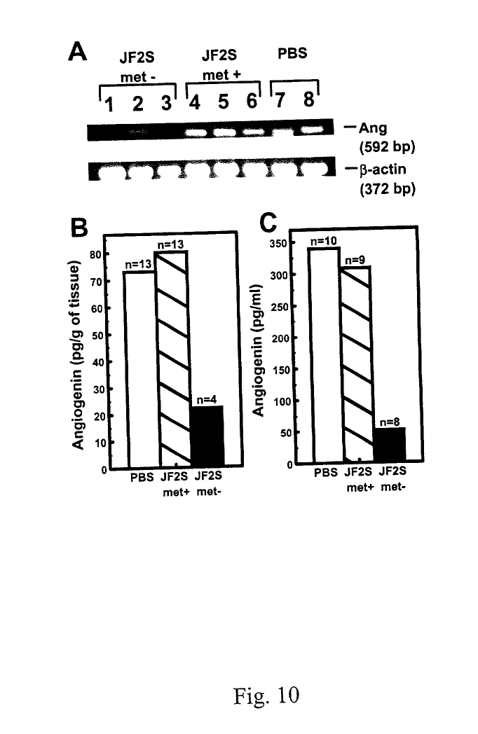

The correlation between ANG quantitation and the presence of metastasis can be also seen in FIG. 10. In this experiment a model of human prostate cancer was employed in which metastatic tumor cells from the human PC-3M cell line were injected into surgically-exposed prostate glands in athymic mice. Untreated control mice so injected will develop primary PC-3M tumors in their prostates with subsequent regional metastasis to the local iliac lymph nodes in a manner similar to the development of metastatic human prostate cancer. In this experiment the mice were treated with either phosphate-buffered saline (PBS) as diluent control or with an ANG antagonist designated JF2S, an antisense directed to ANG which has been shown previously to inhibit the in vitro production of ANG in this and other tumor cell lines (Olson et al., Clin. Cancer Res. In press 2001; and U.S. Pat. No. 6,265,388, hereby incorporated by reference in their entirety for all purposes). At the termination of the experiment on day 33 the primary tumors in the prostates of these mice were excised and total RNA and protein were extracted from the tumors. RT-PCR analysis showed that the expression of ANG was depressed in tumors in which regional iliac lymph node metastases were absent in contrast to tumors derived from PBS-treated, metastasis-harboring controls (FIG. 10A). mRNA levels from tumors derived from JF2S-treated mice in which metastasis developed remained near those of controls. Human ANG protein concentrations in tumor extracts showed a similar pattern (FIG. 10B). While the amount of ANG per gram of tissue extracted from primary tumors of either PBS-treated mice or those mice treated with JF2S in which metastasis was detected was essentially the same (73±18 (SE) vs. 80±24 pg/g of tissue, respectively; P=0.448 by the Wilcoxon rank-sum test), the amount of ANG obtained from tumors of mice successfully protected from metastasis by JF2S was significantly less than either of the former groups (22±1 pg/g of tissue; P=0.0017 and 0.0055, respectively). Serum samples collected at sacrifice were also examined for human ANG concentrations by ELISA (FIG. 10C.). Once again, while ANG concentrations in the sera of control PBS-treated mice or those mice with metastasis that were treated with JF2S were not significantly different (339±30 vs. 308±62 pg/ml, respectively; P=0.656), the level of ANG in sera of metastasis-free mice given JF2S was ˜6-fold less (51±19 pg/ml) than that found in either the PBS control (P=0.00003) or JF2S-treated mice with metastasis (P=0.00099). Thus the presence of metastasis in these mice with primary prostate tumors of human origin could be predicted by quantitation of either the ANG mRNA or ANG protein in the primary prostate tumors or the ANG protein in the sera of the mice.

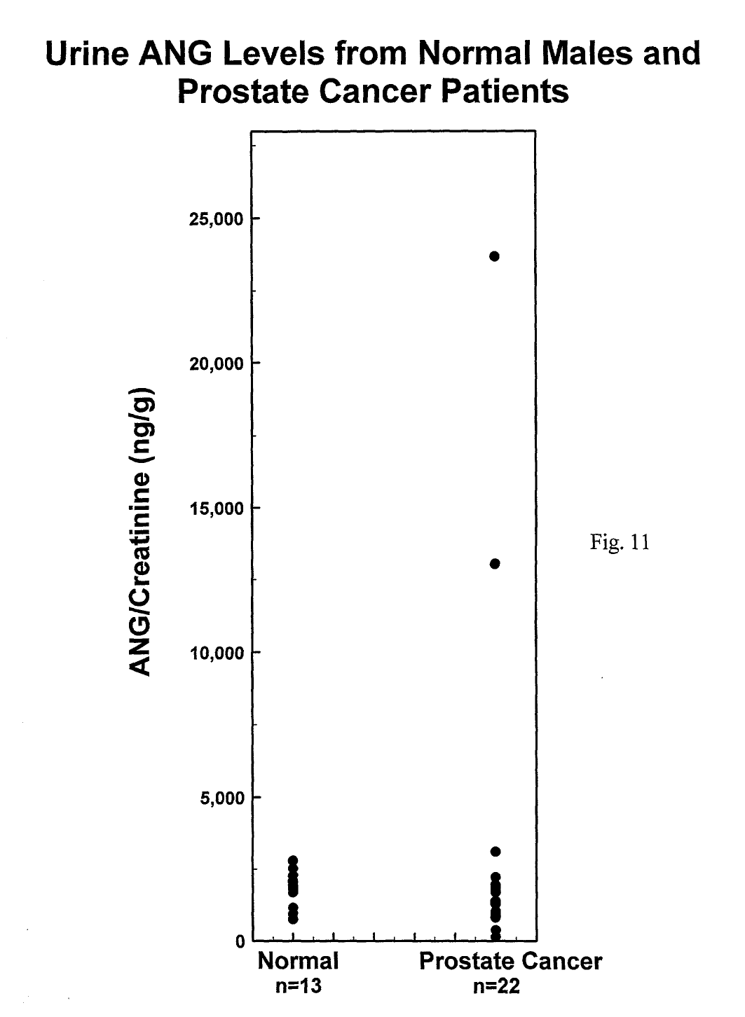

In accordance with preferred embodiments, a method for screening biological samples for ANG is disclosed. More preferably, a method for screening urine samples of men for ANG protein is disclosed. An ELISA assay was used to measure the amount of ANG protein present in the urines of normal men and men with prostate disease. The protocol and methods described by Newton et al., Biochemistry, 35:545-553 (1996), hereby incorporated by reference in its entirety for all purposes, were followed. FIG. 11 shows the screening of urine samples from normal men and men having pathological prostate disease or outcome. The ANG/Creatinine levels in normal men were found to be concentrated within a small range (0-3000 ng/g range). The ANG/Creatinine levels in men having prostate cancer, however, were found to range from almost zero to about 25,000 ng/g. This result is consistent with the observed upregulation of ANG in prostate cancer, as shown in FIG. 6. Therefore, detection of ANG protein may be used as a tool for detection and diagnosis of prostate cancer in men. One skilled in the art would recognize that this screening method could be used in any pathological condition where ANG is upregulated or in metastatic diseases that are characterized by the presence of abnormal amounts of ANG. Therefore, the present invention can be used for detecting metastatic diseases other than prostate cancer.

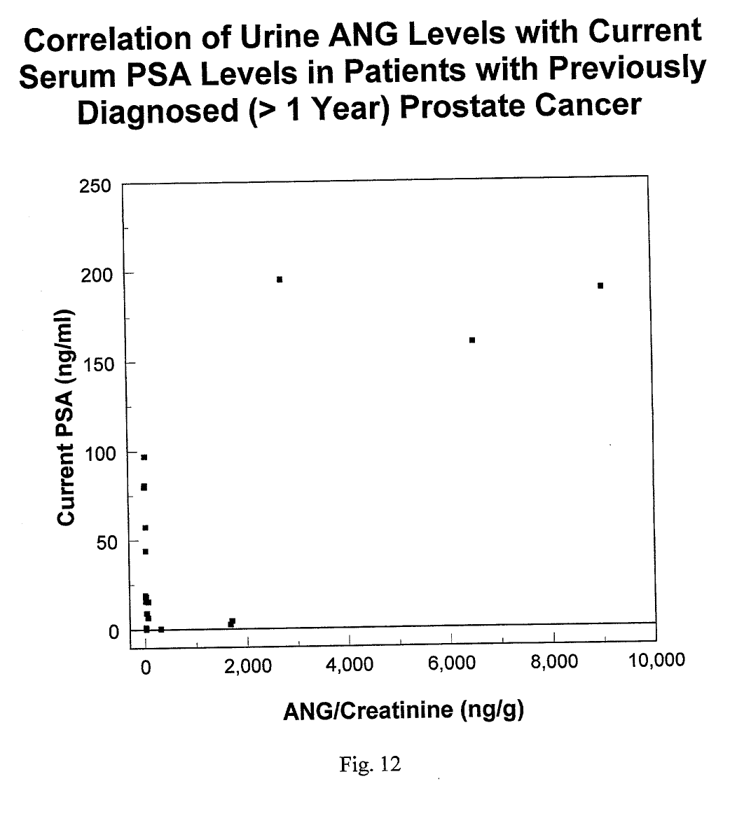

Using the ELISA assay described in FIG. 11, the urine samples from men (n=21) with prostate cancer diagnosed at least one year before urine sample collection were assayed for ANG. These values, expressed as ANG/Creatinine (ng/g) were then compared with the PSA levels (ng/ml) obtained from serum samples taken at the same time or no more than 40 days before the urine samples (FIG. 12). This patient population had received different treatment regimens for prostate cancer, including radical prostatectomy, radiation and antiandrogen drugs. FIG. 12 shows that a correlation exists between the ANG/Creatinine levels in urine and the levels of serum PSA in that the 18 patients with a ANG/Creatinine level below 2000 ng/g also had serum PSA levels below 100 ng/ml, while the 3 patients exhibiting urine ANG/Creatinine above 2000 ng/g had serum PSA levels over 150 ng/ml. Thus screening patients with prostate cancer for urine ANG/Creatinine levels can serve as a means of determining efficacy of treatment and prognosis in a manner similar to a known method, PSA screening.

Methods of the present invention are advantageous for diagnosing the stage and type of metastatic diseases, such as prostate cancer, since the levels of ANG DNA, ANG mRNA or ANG protein reflect the type of prostate cancer, non-metastatic or metastatic, that is present. According to the present invention, a database of the levels of ANG DNA, ANG mRNA or ANG protein associated with known metastatic conditions and the various stages thereof is created. Then tissue or fluid can be tested according to the methods disclosed herein and the amount or level of ANG DNA, ANG mRNA or ANG protein determined can be compared with known values in the database as a method of diagnosing a particular metastatic condition or particular stage of a metastatic condition. It is to be understood that the embodiments of the present invention that have been described are merely illustrative of some of the applications of the principles of the present invention. Numerous modifications may be made by those skilled in the art based upon the teachings presented herein without departing from the true spirit and scope of the invention. Each reference disclosed herein is incorporated by reference in its entirety for all purposes.

What is claimed is:

1. A method for detecting a cancerous condition, comprising:
providing a biological sample;
identifying an amount of angiogenin protein, angiogenin RNA, or angiogenin DNA in the biological sample;
comparing the amount of angiogenin protein, angiogenin RNA, or angiogenin DNA present in the biological sample with known amounts of angiogenin protein, angiogenin RNA, or angiogenin DNA present in cancerous conditions; and
determining existence of a cancerous condition.

2. A method for detecting a metastatic disease, comprising:
providing a biological sample;
identifying an amount of angiogenin protein, angiogenin RNA, or angiogenin DNA included in a biological sample;
comparing the amount of angiogenin protein, angiogenin RNA, or angiogenin DNA present in the biological sample with known amounts of angiogenin protein, angiogenin RNA, or angiogenin DNA present in metastatic diseases; and
determining existence of a metastatic disease.

3. A method for detecting angiogenin DNA, comprising:
providing a biological sample;
isolating DNA in the biological sample;
detecting angiogenin DNA in the isolated DNA.

4. The method of claim 3, wherein said step of detecting angiogenin DNA includes the use of a probe that binds to angiogenin DNA.

5. A method for detecting angiogenin RNA, comprising:
providing a biological sample;
isolating RNA in the biological sample;
detecting angiogenin RNA in the isolated RNA.

6. The method of claim 5, wherein said step of detecting angiogenin RNA includes the use of a probe that binds to angiogenin RNA.

7. A method of detecting angiogenin RNA, comprising:
providing a biological sample;
isolating RNA in the biological sample;
performing RT-PCR on the isolated RNA;
detecting angiogenin RNA.

8. The method of claim 7, wherein said step of detecting angiogenin RNA includes the use of a probe that binds to angiogenin RNA.

9. A method for detecting angiogenin protein comprising:
providing a biological sample;
isolating angiogenin protein in the biological sample;
detecting the isolated angiogenin protein.

10. The method of claim 9, wherein said step of detecting the isolated protein includes the use of a probe that binds to the angiogenin protein.

11. A method for monitoring a cancerous condition or metastatic disease in a subject, comprising:
detecting an amount of angiogenin protein, angiogenin RNA, or angiogenin DNA present in a first biological sample obtained from the subject;
detecting an amount of angiogenin protein, angiogenin RNA, or angiogenin DNA present in a second biological sample from the subject; and
comparing the amount of angiogenin protein, angiogenin RNA, or angiogenin DNA present in the first biological sample with the amount of angiogenin protein, angiogenin RNA, or angiogenin DNA present second sample such that a cancerous condition or metastatic disease is monitored.

12. A method for diagnosing a cancerous condition or a metastatic disease in a subject, comprising:
identifying an amount of angiogenin protein, angiogenin RNA, or angiogenin DNA present in a biological sample obtained from the subject; and
comparing the amount of angiogenin protein, angiogenin RNA, or angiogenin DNA present in the biological sample with known amounts of angiogenin protein, angiogenin RNA, or angiogenin DNA present in cancerous conditions such that a cancerous condition or metastatic disease is diagnosed.

13. A method for prognosing a cancerous condition or a metastatic disease in a subject, comprising:
identifying an amount of angiogenin protein, angiogenin RNA, or angiogenin DNA present in a biological sample obtained from a subject; and
comparing the amount of angiogenin protein, angiogenin RNA, or angiogenin DNA present in the biological sample with known amounts of angiogenin protein, angiogenin RNA, or angiogenin DNA present in cancerous conditions such that a cancerous condition or a metastatic disease is prognosed.

14. A method for assessing the efficacy of a test compound for inhibiting a cancerous condition or a metastatic disease in a subject, comprising comparing:
an amount of angiogenin protein, angiogenin RNA, or angiogenin DNA present in a first biological sample obtained from the subject exposed to or maintained in the test compound with
an amount of angiogenin protein, angiogenin RNA, or angiogenin DNA present in a second biological sample from the subject;
wherein a reduced amount of angiogenin protein, angiogenin RNA, or angiogenin DNA present in the first biological sample relative to the second biological sample, is an indication that the test compound is efficacious for inhibiting a cancerous condition in the subject.

15. A method of assessing the efficacy of a therapy for inhibiting a cancerous condition or a metastatic disease in a subject, comprising comparing:
an amount of angiogenin protein, angiogenin RNA, or angiogenin DNA present in a first biological sample obtained from the subject prior to providing at least a portion of the therapy to the subject with
an amount of angiogenin protein, angiogenin RNA, or angiogenin DNA present in a second biological sample from the subject following providing a portion of the therapy;
wherein a reduced amount of angiogenin protein, angiogenin RNA, or angiogenin DNA present in the first biological sample relative to the second biological sample, is an indication that the therapy is efficacious for inhibiting a cancerous condition or a metastatic disease in the subject.

16. A method of selecting a composition for inhibiting a cancerous condition or a metastatic disease in a subject, comprising:
obtaining a sample comprising cells from the subject;
separately maintaining aliquots of the sample in the presence of a plurality of test compositions;
comparing expression of angiogenin protein, angiogenin RNA, or angiogenin DNA in each of the aliquots, and
selecting one of the test compositions which induces a reduced amount of angiogenin protein, angiogenin RNA, or angiogenin DNA in the aliquot containing that test composition, relative to other test compositions.

17. A method for treating a cancerous condition or a metastatic disease in a subject, comprising administering to the subject a compound such that the amount of angiogenin protein, angiogenin RNA, or angiogenin DNA in the patient is reduced.

18. A kit for assessing whether a subject is afflicted with a cancerous condition or a metastatic disease, wherein the kit comprises reagents for assessing the amount of angiogenin protein, angiogenin RNA, or angiogenin DNA.
